(19) United States
(12) Patent Application Publication
(10) Pub. No.: US 2012/0328516 A1
(43) Pub. Date: Dec. 27, 2012

(54) METHOD OF RADIOTHERAPY USING A RADIOLABELLED RGD PEPTIDE

(76) Inventors: BRIAN J. MCPARLAND, Amersham (GB); JULIAN GOGGI, Singapore (SG)

(21) Appl. No.: 13/533,316

(22) Filed: Jun. 26, 2012

Related U.S. Application Data
(63) Continuation-in-part of application No. PCT/US2010/062279, filed on Dec. 29, 2010.
(60) Provisional application No. 61/290,926, filed on Dec. 30, 2009.

(51) Int. Cl. A61K 51/08; A61P 35/00

(52) U.S. Cl. 424/1.69

(57) ABSTRACT

The present invention relates to a method of radiotherapy, in particular image-guided radiotherapy (IGRT), where radiolabelled RGD peptides are used to provide a tumour angiogenesis volume. Also provided are methods of determination of the gross tumour volume as well as methods of radiotherapy monitoring using such peptides.

This application is a continuation-in-part application of PCT/US2010/062279 filed Dec. 29, 2010 published as WO2011/082191 on Jul. 7, 2011 which claims priority to U.S. application No. 61/290,926 filed Dec. 30, 2009, the entire disclosure of each of which is hereby incorporated by reference.

### FIELD OF THE INVENTION

The present invention relates to a method of radiotherapy, in particular image-guided radiotherapy (IGRT), where radiolabelled RGD peptides are used to provide a tumour angiogenesis volume. Also provided are methods of determination of the gross tumour volume as well as methods of radiotherapy monitoring using such peptides.

### BACKGROUND TO THE INVENTION

Modern external beam radiotherapy of neoplasia relies upon rapidly-developing technologies in order to maximise cure rates (through maximisation of tumour control probability) and to minimise complication rates (through minimisation of normal tissue irradiation). The technologies include the use of imaging coincident with treatment delivery (e.g., through electronic portal imaging devices) in image-guided radiotherapy (IGRT) and multi-leaf collimators to allow complex treatment portals with variable beam intensities or intensity-modulated radiotherapy (IMRT).

These approaches require, during the radiotherapy planning (RTP) process, the accurate definition of a number of concentric volumes defined in ICRU Reports 50 and 62 (International Commission on Radiation Units and Measurements. Prescribing, recording and reporting photon beam therapy (1993)]. The first is that of the gross tumour volume (GTV) based upon anatomical morphology derived from, for example, CT or a fluoroscopic/radiographic image from a treatment simulator. Following, the radiation oncologist expands upon this to define the clinical treatment volume (CTV) based upon additional clinical data and experience. Finally, a planning treatment volume (PTV) is defined around the CTV in order to account for organ and patient motion and setup errors.

Functional imaging can detect regions of biological activity not possible with conventional radiological imaging, since that is based on anatomical imaging alone. Consequently, functional imaging can lead to modifications of the GTV defined solely on the basis of anatomical imaging. Clinical studies of the use of PET using fluorine-18 FDG in RTP of non-small-cell lung cancer, oesophageal cancer and head and neck cancer have shown that the inclusion of PET imaging in the planning process has resulted in changes to the GTV definition [Turin et al, Br. J. Radiol, S27-S35 (2006)] and Bradley et al Int. J. Radiat. Oncol. Biol. Phys., 59: 78-86 (2004)].

The use of PET in radiotherapy planning has also been shown to reduce the inter-operator variability in defining the GTV. The use of PET-CT in RTP is based almost exclusively based upon ¹⁸F-FDG, which is a general marker of metabolism.

There is therefore still a need to further improve tumour radiotherapy, whereby irradiation is targeted successfully to tumorous or potentially tumorous cells, and side effects dues to unnecessary irradiation of normal tissue are minimised. This is to maximise the therapeutic ratio which is the ratio of the tumour control probability to the normal tissue complication rate. See introduction by Perez C A and Brady L W (Eds), Principles and Practice of Radiation Oncology, Philadelphia: JB Lippincott Co (1987)].

### THE PRESENT INVENTION

The present inventors have found that radiolabelled Arg-Gly-Asp (RGD) peptides provide additional relevant information useful in tumour radiotherapy. Whereas ¹⁸F-FDG indicates the presence of metabolically-active tumour cells, where glucose metabolism is occurring, radiolabelled RGD peptides indicate the presence of αvβ3 or αvβ5 integrins, which can be associated with both expression of the integrins by the tumour cells themselves or the presence of angiogenesis.

Importantly, the angiogenic border of a tumour is not entirely coincident with the border of glucose-metabolising detectable with ¹⁸F-FDG. In fact, the tumour volume which uptakes the radiolabelled RGD peptide was found to be greater than the tumour volume as measured by ¹⁸F-FDG. Thus, certain tumour cells are relatively inactive metabolically inactive and/or undergoing vascularisation appear to be occult to ¹⁸F-FDG. The present invention indicates that the radiolabelled RGD peptides, by indicating the presence of αvβ3 or αvβ5 integrins, possibly denote areas in the neoplasia which contain dormant tumour cells and/or angiogenesis that may become viable tumour but which cannot be detected by ¹⁸F-FDG. This would lead to important changes in the GTV definition, to ensure that such tumour cells receive appropriate irradiation during the radiotherapy. Modern-day intensity-modulated radiation therapy (IMRT) and image-guided radiation therapy (IGRT) seek to restrict the volume irradiated to that of the tumour only, so as to allow an increase in radiation absorbed dose to the tumour so as to elevate the tumour control probability whilst restricting the radiation-induced complication rate of normal tissue in the vicinity of the tumour.

### DETAILED DESCRIPTION OF THE INVENTION

In a first aspect, the present invention provides a method of tumour radiotherapy of a mammalian subject having a tumour, where said method comprises:
(i) determination of the volume of angiogenesis of said tumour by in vivo imaging of said subject previously administered with a radiopharmaceutical comprising radiolabelled RGD peptide, wherein said RGD peptide targets the integrin αvβ3 or αvβ5 receptor;
(ii) using the determination from step (i) to define a gross tumour volume;
(iii) using the gross tumour volume from step (ii) to define a planned treatment volume;
(iv) carrying out radiotherapy on the planned treatment volume of step (iii).

The term “mammalian subject” refers to the mammalian body in vivo, preferably the human body in vivo, more preferably the intact human body in vivo.

The term “radiotherapy” refers to therapy wherein ionizing radiation is used to control or kill cells of undesirable growth. Such techniques are known in the art, and are described e.g. by S. H. Levitt et al (Eds) [Technical Basis of Radiation Therapy: Practical Clinical Applications, 5^{th} edition, Springer (2011)], and C.A. Perez et al [Principles and Practice of Radiation Oncology, JB Lippincott (1987)]. The radiation may be provided from a source generating a beam of radiation, such as an accelerator (e.g., a synchrotron or cyclotron) or radiation source (eg. a radioactive source). A preferred such technique is external beam radiation therapy, which includes conventional external beam radiotherapy, as well as stereotactic radiotherapy, intensity-modulated radiation therapy and image-guided radiation therapy.

The method of the present invention is thus a method of image-guided radiotherapy, also known as “IGRT”. Such IGRT includes 3-dimensional conformal radiotherapy (3DCRT), conformal radiotherapy (CRT) and intensity modulated radiotherapy (IMRT). Further details of IGRT are provided by J. D. Bourland (Ed) [Image-Guided Radiation Therapy, Taylor & Francis (2012)] and Mundt and Roeske [Image-Guided Radiation Therapy (IGRT): A Clinical Perspective, McGraw-Hill Medical, (2011)].

The term “radiopharmaceutical” has its conventional meaning, and refers to a radioactive compound suitable for in vivo mammalian administration for use in diagnosis or therapy. Suitable radiopharmaceuticals for use in the present invention are those suitable for medical imaging—the radiopharmaceutical itself is not part of the radiotherapy.

The term “radiolabelled” means that either the RGD peptide comprises the radioisotope (eg. a ¹¹C label as an intrinsic part of the chemical structure), or the radioisotope is attached as an additional species. The radioisotope is preferably suitable for radiopharmaceutical imaging using SPECT or PET. Suitable positron-emitting radioisotopes for PET include: ¹⁸F, ¹¹C, ¹³N, ⁷⁵Br, ⁷⁶Br, ¹²⁴I, ⁶⁴Cu, ⁴⁸V, ⁵²Fe, ⁵⁵Co, ^{94m}Tc or ⁶⁸Ga. Preferred such PET isotopes are: ¹⁸F, ¹¹C, ¹²⁴I, ⁶⁴Cu, and ⁶⁸Ga, more preferably ¹⁸F, ¹²⁴I and ⁶⁸Ga, most preferably ¹⁸F. Suitable gamma-emitting radioisotopes for SPECT include: ^{99m}Tc, ¹²³I, ¹¹¹In, ^{113m}In, ⁶⁷Cu or ⁶⁷Ga. Preferred such SPECT isotopes are ^{99m}Tc and ¹²³I, more preferably ^{99m}Tc.

The term “RGD peptide” has its conventional meaning in the field of radiopharmaceuticals and radiotherapy, and refers to peptides which include the motif Arg-Gly-Asp. The RGD peptides of the present invention preferably comprise 4 to 30, more preferably 5 to 20, most preferably 5 to 15 amino acids. Suitable RGD peptides are those with selectivity for the integrin αvβ3 or αvβ5 receptor, preferably the αvβ3 receptor. Such peptides and integrin αvβ3 imaging agents derived therefrom are known in the art, and are described by: Beer et al [Methods Mol. Biol., 680, 183-200 (2011); Theranostics, 1, 48-57 (2011)]; Morrison et al [Theranostics, 1, 149-153 (2011)]; Zhou et al [Theranostics, 1, 58-82 (2011)] and Auzzas et al [Curr. Med. Chem., 17(13), 1255-1299 (2010)].

The term “gross tumour volume” (GTV) has its conventional meaning in the field of radiotherapy, and corresponds to the gross palpable, visible or clinically-demonstrable location and extent of the malignant growths. The GTV delineates the clinically-demonstrable extent of malignant disease through palpation or imaging. See ICRU Report 50 (1993) and 62 (1993), where ICRU is the International Commission on Radiation Units and Measurements.

The “clinical target volume” (CTV) is the volume containing the GTV and subclinical (occult) malignant disease defined at a certain probability level. The CTV includes the GTV but has a greater margin in order to include occult disease that is believed to be present with a pre-determined probability.

The “planning tumour volume” (PTV) surrounds the CTV with a margin sufficient to compensate for uncertainties in the delivery of treatment. As such, the PTV is geometric and compensates for physiological movement and uncertainties in alignment of the patient anatomy with the radiation beam. The PTV includes the CTV but has a greater margin to account for anatomical or physiological movements and uncertainties in patient set-up. See ICRU Report 50 (1993) and Report 62 (1993), where ICRU is the International Commission on Radiation Units and Measurements. Further details of how gross tumour volume and planned tumour volume are used in radiotherapy are described by Lecchi et al [Eur. J. Nucl. Med. Mol. Imaging, 35, 821-837 (2008)].

It is believed that imaging with the radiolabelled RGD peptides of the present invention could be used to determine the GT,V but also to differentiate more clearly between the GTV and CTV by making evident the extent of disease that would otherwise be sub-clinical.

The terms “comprises” or “comprising” have their conventional meaning throughout this application and imply that the composition must have the components listed, but that other, unspecified compounds or species may be present in addition. The term ‘comprising’ includes as a preferred subset “consisting essentially of” which means that the composition has the components listed without other compounds or species being present.

Preferred Features.

In the first aspect, the RGD peptide is preferably radiolabelled with ¹⁸F or ^{99m}Tc, more preferably ¹⁸F. The radiolabelled RGD peptide is preferably chosen from:
(i) ¹⁸F-fluciclatide;
(ii) ^{99m}Tc-maraciclatide;
(iii) ¹⁸F-galacto-RGD; or
(iv) ¹⁸F-RGD-K5.

The term “[¹⁸F]-fluciclatide” refers to the compound of Formula I:

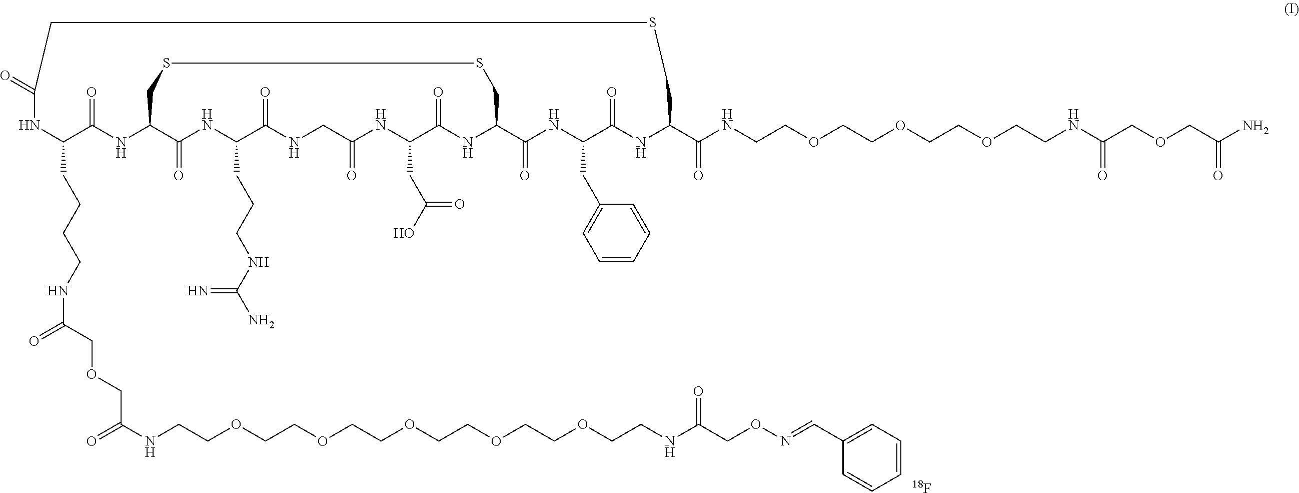

Fluciclatide (¹⁸F) is the recommended INN (US Approved Name) for [¹⁸F]-AH111585. The chemical structure of Formula (I) shows the oxime ether as the trans isomer. The term “[¹⁸F]-fluciclatide” as used herein encompasses a mixture of the cis and trans isomers, as well as substantially pure separated cis-isomer or trans-isomer. [¹⁸F]-fluciclatide is prepared by radiofluorination of Precursor 1 using [¹⁸F]-fluorobenzaldehyde:

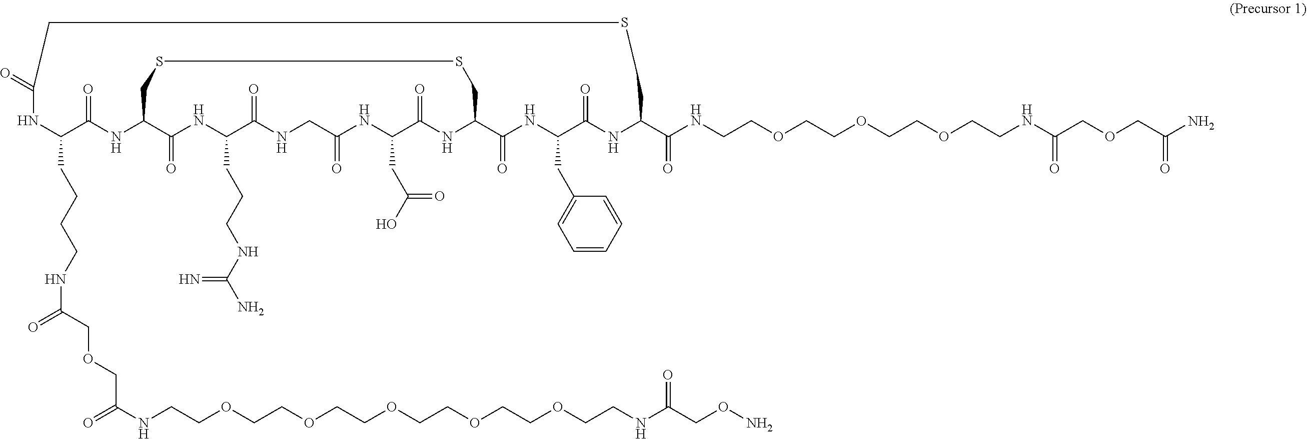

Precursor 1 is non-radioactive. It can be prepared as described by Indrevoll et al [Bioorg. Med. Chem. Lett., 16, 6190-6193 (2006)].

The term “maraciclatide” refers to the compound known in the scientific literature as NC100692 [D. Edwards et al, Nucl. Med. Biol., 35, 365-375 (2008)]. The chemical name is: 1,5-pentanedioic acid-(5-[2-hydroxyimino-1,1-dimethyl-propylamino]-3-(2-[2-hydroxyimido-1,1-dimethyl-propylamino]-ethyl)-pentyl)-amide 5-[13-benzyl-19-carboxymethyl-25-(3-guanidino-propyl)-10-(4,7,10,16-tetraoxa-14,18-dioxo-1,13,19-triazanonadecyl)-carbamoyl-3,6,12,15,18,21,24,27-octaoxo-8,29,30-trithia-2,5,11,14,17,20,23,26-octaaza-bicyclo[14.11.4]hentriacont-4-yl]pentyl-amide.

The chemical structure of maraciclatide is as follows:

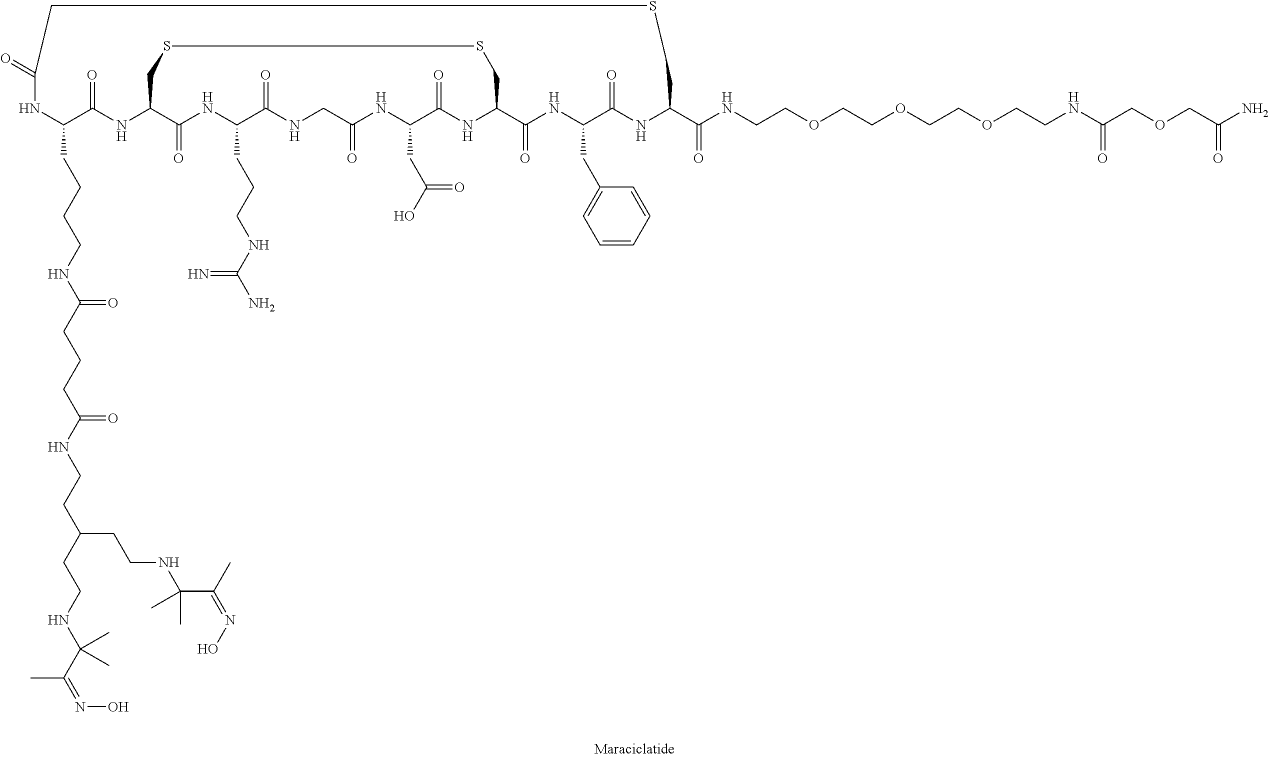

In ^{99m}Tc-maraciclatide, the ^{99m}Tc radioisotope is chelated by the diaminedioxime chelator of maraciclatide.

¹⁸F-galacto-RGD refers to ¹⁸F-labelled galactosyl-RGD as described by Laitenen et al [Circ. Cardiovasc. Imaging, 2, 331-338 (2009)] and Beer et al [Clin. Cancer Res., 13(22), 6610-6616 (2007) and J. Nucl. Med., 46(8), 1333-1341 (2005)]. It can be prepared as described therein.

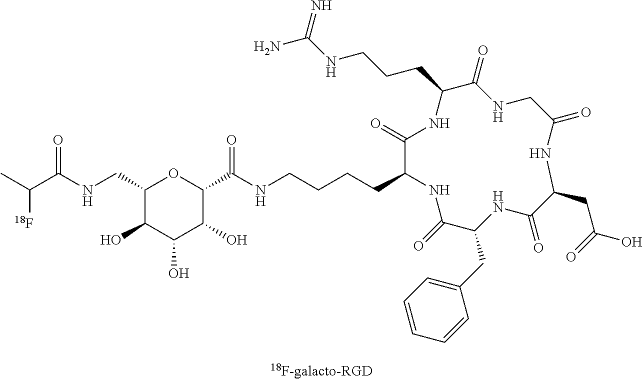

¹⁸F-RGD-K5 has the chemical structure shown:

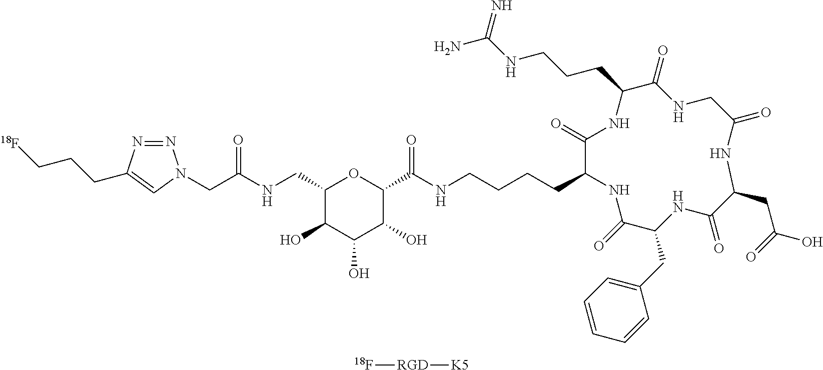

Further details of ¹⁸F-RGD-K5, including its synthesis are described by Doss et al [J. Nucl. Med., 53(5), 787-795 (2012)], and Walsh et al [Chimia, 64(1/2), 29-33 (2010)].

The radiolabelled RGD peptide of the first aspect is more preferably ¹⁸F-fluciclatide or ^{99m}Tc-maraciclatide, and is most preferably ¹⁸F-fluciclatide.

The production of [¹⁸F]fluoride suitable for radiopharmaceutical applications is well-known in the art, and has been reviewed by Hjelstuen et al [Eur. J. Pharm. Biopharm., 78(3), 307-313 (2011)], and Jacobson et al [Curt Top. Med. Chem., 10(11), 1048-1059 (2010)].

In a second aspect, the present invention provides a method of determination of the gross tumour volume of a tumour of a mammalian subject having a tumour, where said method comprises:
(i) determination of the volume of angiogenesis of said tumour by in vivo imaging of said subject previously administered with a radiopharmaceutical comprising a radiolabelled RGD peptide, wherein said RGD peptide targets the integrin αvβ3 or αvβ5 receptor;
(ii) using the determination from step (i) to define a gross tumour volume.

Suitable and preferred aspects of the subject and radiolabelled RGD peptide in the second aspect are as described in the first aspect (above).

In a third aspect, the present invention provides a method of monitoring the tumour radiotherapy of a mammalian subject having a tumour, which comprises:
(a) carrying out the tumour radiotherapy method of the first aspect;
(b) at intervals following the radiotherapy of step (a), repeating the determination of the volume of angiogenesis of said tumour as defined in step (i) of the first aspect;
(c) comparing the volume of tumour angiogenesis of step (b) with the volume of tumour angiogenesis prior to radiotherapy irradiation of the tumour, to see if the radiotherapy has reduced the size of the volume of angiogenesis;
(d) optionally repeating steps (a)-(c).

Suitable and preferred aspects of the subject, radiotherapy and radiolabelled RGD peptide in the third aspect are as described in the first aspect (above).

### DESCRIPTION OF THE FIGURES

FIG. 1 shows co-registered contrast-enhanced CT and PET images of a necrotic lymph node metastasis for a primary breast cancer. The emission image was obtained for ^{99m}Tc-maraciclatide. The necrotic centre in the CT image which corresponds to no uptake of the agent in the SPECT image. The region surrounding the necrotic centre due to angiogenesis and other expressions of alphavbeta3/5 integrins is highlighted (uptake in the sternum is concluded to be due to bone marrow uptake).

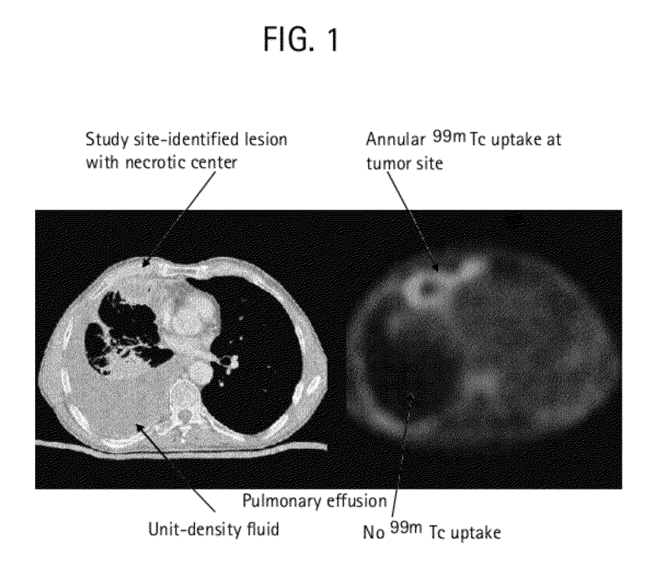

FIG. 2 shows co-registered contrast-enhanced CT and PET images of a necrotic lymph node metastasis for a primary breast cancer. The emission image was obtained using ¹⁸F-fluciclatide. The necrotic centre in the CT image which corresponds to no uptake of the agent in the PET image. The region surrounding the necrotic centre due to angiogenesis and other expressions of alphavbeta3/5 integrins is highlighted (uptake in the sternum is concluded to be due to bone marrow uptake).

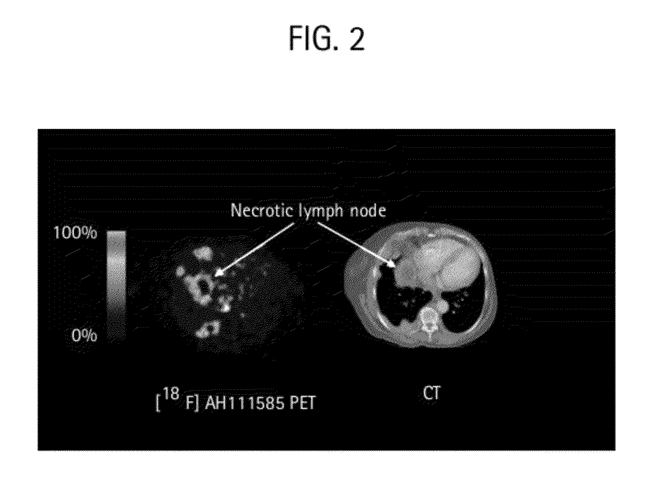

FIGS. 3A and 3B show co-registered coronal micro-PET/CT images of the same C57/BI6 mouse bearing a subcutaneous U87 tumour on its left side. The emission image in 3A is that of ¹⁸F-FDG and that in 3B is of ¹⁸F-fluciclatide. Note the difference in tumour uptake volumes between the two images.

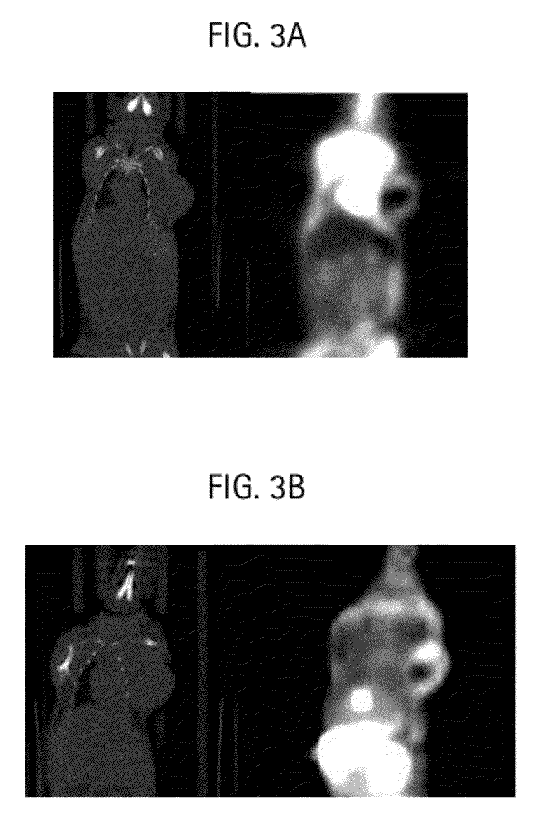

The invention is illustrated by the non-limiting Examples detailed below. Example 1 provides.

Abbreviations.

CT: computed axial tomography,

CTV: clinical treatment volume,

FDG: fluorodeoxyglucose,

GTV: gross tumour volume,

IGRT: image-guided radiotherapy,

IMRT: intensity-modulated radiotherapy,

PET: positron emission tomography,

PTV: planning treatment volume,

RTP: radiotherapy planning.

1. A method of tumour radiotherapy of a mammalian subject having a tumour, where said method comprises:
(i) determination of the volume of angiogenesis of said tumour by in vivo imaging of said subject previously administered with a radiolabelled RGD peptide, wherein said RGD peptide targets the integrin αvβ3 or αvβ5 receptor;
(ii) using the determination from step (i) to define a gross tumour volume;
(iii) using the gross tumour volume from step (ii) to define a planned treatment volume;
(iv) carrying out radiotherapy on the planned treatment volume of step (iii).

2. The method of claim 1, where the radiotherapy is external beam radiation therapy.

3. The method of claim 1, where the RGD peptide is radiolabelled with ¹⁸F or ^{99m}Tc.

4. The method of claim 3, where the radiolabelled RGD peptide is chosen from:
(i) ¹⁸F-fluciclatide;
(ii) ^{99m}Tc-maraciclatide;
(iii) ¹⁸F-galacto-RGD; or
(iv) ¹⁸F-RGD-K5.

5. The method of claim 4, where the radiolabelled RGD peptide is chosen from:
(i) ¹⁸F-Fluciclatide; or
(ii) ^{99m}Tc-maraciclatide.

6. A method of determination of the gross tumour volume of a tumour of a mammalian subject having a tumour, where said method comprises:
(i) determination of the volume of angiogenesis of said tumour by in vivo imaging of said subject previously administered with a radiolabelled RGD peptide, wherein said RGD peptide targets the integrin αvβ3 or αvβ5 receptor;
(ii) using the determination from step (i) to define a gross tumour volume.

7. The method of claim 6, where the RGD peptide is radiolabelled with ¹⁸F or ^{99m}Tc.

8. The method of claim 7, where the radiolabelled RGD peptide is chosen from:
(i) ¹⁸F-fluciclatide;
(ii) ^{99m}Tc-maraciclatide;
(iii) ¹⁸F-galacto-RGD; or
(iv) ¹⁸F-RGD-K5.

9. The method of claim 8, where the radiolabelled RGD peptide is chosen from:
(i) ¹⁸F-Fluciclatide; or
(ii) ^{99m}Tc-maraciclatide.

10. A method of monitoring the tumour radiotherapy of a mammalian subject having a tumour, which comprises:
(a) carrying out the tumour radiotherapy method as defined in claim 1;
(b) at intervals following the radiotherapy of step (a), repeating the determination of the volume of angiogenesis of said tumour as defined in step (i) of claim 1;
(c) comparing the volume of tumour angiogenesis of step (b) with the volume of tumour angiogenesis prior to radiotherapy irradiation of the tumour, to see if the radiotherapy has reduced the size of the volume of angiogenesis;
(d) optionally repeating steps (a)-(c).

11. The method of claim 10, where the radiotherapy is external beam radiation therapy.

12. The method of claim 10, where the RGD peptide is radiolabelled with ¹⁸F or ^{99m}Tc.

13. The method of claim 12, where the radiolabelled RGD peptide is chosen from:
(i) ¹⁸F-fluciclatide;
(ii) ^{99m}Tc-maraciclatide;
(iii) galacto-RGD; or
(iv) ¹⁸F-RGD-K5.

14. The method of claim 13, where the radiolabelled RGD peptide is chosen from:
(i) ¹⁸F-Fluciclatide; or
(ii) ^{99m}Tc-maraciclatide.
